(19) United States
(12) Patent Application Publication
(10) Pub. No.: US 2020/0155014 A1
(43) Pub. Date: May 21, 2020

(54) SYSTEM AND METHOD FOR DEPLOYMENT OF AN IMPLANTABLE DEVICE HAVING AN ATTACHMENT ELEMENT AND METHODS OF MONITORING PHYSIOLOGICAL DATA USING MULTIPLE SENSOR DEVICES

(71) Applicant: MICROTECH MEDICAL TECHNOLOGIES LTD., Tel Aviv (IL)

(72) Inventors: Amir ARTHUR, Tel Aviv (IL); Nadav SHARON, Herzliya (IL); Amir KOHEN, Tel Aviv (IL); Amir HAREL, Tel Aviv (IL); Erez JANAI, Petach Tikya (IL)

(21) Appl. No.: 16/686,700

(22) Filed: Nov. 18, 2019

Related U.S. Application Data
(60) Provisional application No. 62/769,137, filed on Nov. 19, 2018.

(51) Int. Cl. A61B 5/0215; A61B 5/00; A61B 8/12; A61B 8/08; A61B 17/34

(52) U.S. Cl. CPC A61B 5/0215; A61B 5/6869; A61B 8/12; A61B 2560/063; A61B 8/0883; A61B 5/6882; A61B 17/3423; A61B 8/488

(57) ABSTRACT

Systems and methods for deployment and implantation of an implantable device having an attachment element directly in a luminal wall or tissue to monitor or detect physiological conditions. In an embodiment of the invention, a device is positioned at one or more target locations in the body to enable a medical professional to obtain physiological information for the target site(s). The invention also provides novel methods of monitoring physiological conditions using multiple sensors via techniques such as ultrasound with frequency separation or spatial separation.

This application claims the priority benefit of U.S. provisional patent application Ser. No. 62/769,137, filed Nov. 19, 2018, the contents of which are incorporated herein by reference in their entirety.

### FIELD OF THE INVENTION

The present invention relates to a system and method for deployment and implantation of one or more implantable devices directly in a luminal wall or tissue to monitor or detect physiological conditions, e.g., of a body. In particular, the deployment system includes a cannula, a deployment rod, and a deployment assembly to which the implantable device is releasably attached. The deployment assembly includes a shroud which releases the device upon secure positioning of the device in a target location. The device includes an attachment element that is secured within a luminal wall or tissue to thereby position the device against the luminal wall or tissue.

### BACKGROUND

Deployment systems are used to, e.g., embed implantable devices within a lumen of the body. Generally, a deployment system comprises a catheter, an implantable device, and an element for releasing the device at a target location, for example, described in U.S. Pub. No. 2003/0125790 and U.S. Pub. No. 2008/0071248. The catheter houses the deployment system and permits the system to be advanced to the target location, where the device is released. The device remains within the body to perform its intended function after the deployment system is retracted from the body.

Implantable hemodynamic monitoring (IHM), temperature monitoring, and chemical monitoring are illustrative examples of types of monitoring with implantable sensors that have been shown to be helpful in improving the health and quality of life of patients. Several recent studies have shown that monitoring patients with congestive heart failure can improve their well-being.

Importantly, the device must be securely attached to the target location before the deployment system releases the device. A device which is not securely embedded may become dislodged and pose serious risks to the patient, especially if the device begins to migrate from the implantation site. An insufficiently secured device that circulates in the body may cause serious injuries, including an acute myocardial infarction, a stroke, or organ failure. There is therefore a need for a deployment system that assures secure deployment of the device in the body prior to retraction of the deployment system.

Sensors for monitoring a patient's cardiovascular system have been previously placed in a single location, typically in either the pulmonary artery or the left atrium. However, it has been found that monitoring a patient's cardiovascular system in more than one location can be useful for obtaining additional information about the patient's hemodynamics. This added information can assist the clinician in diagnosing, monitoring and treating medical problems that cannot be diagnosed or monitored using a single sensor, and better clinical prognosis can be achieved for medical conditions. Possible implantation sites for two sensors for monitoring a patient's hemodynamics could include (but are not limited to) the left atrium, right atrium, pulmonary artery, left ventricle, right ventricle. jugular vein, vena cava, carotid artery, vertebral artery, portal vein, hepatic vein, intra-cranial, and intra-ocular regions.

A system that is capable of directly, reliably and securely position a device would reduce the complexities of such a procedure and the need for post-operative treatments, providing favorable outcomes to both the physician and the patient.

A need therefore exists for a deployment system that would allow for direct, safe and secure implantation of one or more devices into the body, at one or more separate implantation locations.

### SUMMARY OF THE INVENTION

One aspect of the present invention relates to a system and method for deployment and secure positioning of an implantable device directly in a luminal wall or tissue to monitor or detect physiological conditions, e.g., of a body. In particular, the deployment system may include a cannula, a deployment rod having a proximal end and a distal end, and a deployment assembly disposed at the distal end of the deployment rod to which the device is releasably attached. The deployment assembly includes a shroud to release the device upon its secure positioning. The device includes an attachment element that is secured within a luminal wall or tissue by advancing (i.e. a forward movement) the deployment rod to thereby position and implant the device against the luminal wall or tissue. In a further embodiment. the deployment assembly may also include a release member, and the shroud may be separated from the device upon disengagement of the release member.

Another aspect of the present invention is directed to a deployment system which includes a cannula, a deployment rod having a proximal end and a distal end, and a deployment assembly to which an implantable device is releasably attached. The device comprises an attachment element for positioning at a target location. The deployment assembly includes a shroud to release the device upon its secure positioning. The shroud is releasably attached to the device. In a further embodiment, the deployment assembly may also include a release member, and the shroud may be detached from the device upon disengagement of the release member. The deployment system may be configured to implant a single device at a target location, or the deployment system may be configured to implant two or more devices at one or more desired target locations during a single procedure. Two or more deployment systems may also be configured such that each deploys an device at a desired target location(s) during a single procedure using the principles of the present invention.

The device includes an attachment element that is secured within a luminal wall or tissue by advancement, for example, by pushing or rotating of the deployment rod. The device may further include a locking plate which may be affixed to a proximal side of a base. The attachment element may be affixed to a distal side of the base which also has a proximal side and a thickness therebetween. The base may comprise a plurality of cutout portions, and one or more of the plurality of cutout portions may comprise a different shape than the other cutout portions.

The shroud may include a plurality of axial securement tabs which may be configured to engage the locking plate of the device thereby preventing axial motion of the device with respect to the deployment assembly. The shroud may also comprise a plurality of circumferential securement tabs which may engage the locking plate to prevent lateral motion of the device.

A method according to another aspect of the invention provides for deploying an device at a target site using a deployment system comprising a cannula and a deployment rod. The device may be releasably attached to a deployment assembly disposed at the distal end of the deployment rod, and the device comprises an attachment element. The deployment assembly may comprise a shroud. The method comprises advancing the deployment system to a target site such that the attachment element of the device is in contact with the target site. The method further includes securely embedding the attachment element into the target site using the deployment rod. After the device is securely embedded in the target site, disengagement of the device from the shroud occurs. The shroud remains affixed to the deployment rod after the device is disengaged from the deployment system. The deployment rod, shroud, and cannula are then withdrawn, thereby separating the device from the shroud. The step of securely embedding the attachment area in the target site may comprise advancing or rotating the deployment rod. In a further embodiment, the deployment assembly may comprise a release member and the step of disengaging the device from the shroud may comprise disengaging the release member to thereby detach a safety mechanism in the form of a lock assembly. The step of disengaging the release member may comprise administering a pull force or other action to the release member to detach the lock assembly.

The method of implanting a device at a target site may be used to implant a single device, or the method may be used to implant two or more devices at a target location. The target location may be a single location in a patient's body, for example, a chamber of the heart, or there may be two or more target locations at different locations within an organ or nearby tissues, for example, both ventricles or atria of the heart, or one ventricle and one atrium of the heart, or combinations thereof. When the target location is two or more distinct sites, one or more sensors may be positioned at each separate site in the body. Depending on the clinician's recommendation, multiple sensors can be implanted during a single procedure or over multiple procedures. Positioning of multiple sensors during a single procedure has the advantage of avoiding multiple invasive surgical sessions which can increase chances of negative patient outcomes.

Another aspect of the present invention is directed to a method of monitoring physiological data. The inventive method comprises deploying two or more small sensors at a target location in a patient's body; and monitoring physiological data from each sensor, for example, using a technique such as frequency separation or spatial separation. The sensors may monitor a physiological property such as blood pressure, or another property in the body. The sensors may monitor the same physiological property or different physiological properties.

The physiological data may be monitored using an imaging technique such as continuous wave Doppler ultrasound imaging. The target locations in the body may be the left and right atria, the left and right ventricles, or an atrium and a ventricle, or another location in the body. The method may further comprise implanting a third (or additional) small sensor at a target location in the patient's body, and monitoring physiological data from the third sensor using frequency separation or spatial separation.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present invention will be understood and appreciated more fully from the following detailed description taken in conjunction with the drawings in which:

FIG. 1 illustrates a block diagram of a deployment system of the present invention.

FIG. 2 illustrates implantable devices having attachment elements in the form of a barb.

FIG. 2A illustrates an implantable device having an attachment element in the form of a coil.

FIG. 2B illustrates an exploded view of an device having an attachment element in the form of a coil.

FIG. 3 illustrates a deployment assembly releasably attached to an implantable device.

FIG. 4A illustrates a first step in releasing the device from a deployment assembly.

FIG. 4B illustrates a second step in releasing the device from a deployment assembly.

FIG. 5A illustrates a cross-sectional view of a deployment assembly attached to an device and releasably coupled to a release wire assembly.

FIG. 5B illustrates a top view of a deployment assembly attached to a device and releasably coupled to a release wire assembly.

FIG. 6 illustrates separate distal and proximal sub-assemblies in an embodiment of a deployment system.

FIG. 7A illustrates a perspective view of a proximal sub-assembly of a deployment system in an embodiment of the invention.

FIG. 7B illustrates an exploded view of the proximal sub-assembly of FIG. 7A.

FIG. 8A shows a first sensor deployed in the right atrium of the heart and a second sensor deployed in the left atrium of the heart in an embodiment of the invention.

FIG. 8B shows a first sensor deployed in the right ventricle of the heart and a second sensor deployed in the left ventricle of the heart, in an embodiment of the invention.

FIG. 8C shows a first sensor deployed in the right atrium of the heart, a second sensor deployed in the left ventricle of the heart on the ventricular septum, and a third sensor deployed in the left atrium of the heart, in an embodiment of the invention.

FIG. 9A is an ultrasound image of two sensors (Sensor 1 and Sensor 2) which are about 1 cm apart and seen as two colored “comet tails” in color Doppler mode obtained using a standard ultrasound imager.

FIG. 9B shows the spectral response of Sensor 1 from FIG. 9A via continuous wave Doppler mode.

FIG. 9C shows the spectral response of Sensor 2 from FIG. 9A via continuous wave Doppler mode in which the beam focus was shifted using a phased-array transducer.

### DETAILED DESCRIPTION OF THE INVENTION

The invention generally relates to systems and methods for direct deployment of a device in the body. In particular, the systems and methods relate to securely implanting a device in the body to monitor or detect a physical, chemical, or biological characteristic. For example, the device may be used to monitor bodily characteristics such as (but not limited to) blood pressure, flow, viscosity, shear rate, shear stress, temperature, glucose levels, calcium levels, electrical conductivity, and electrical potential in a location in the body such as, e.g., inside a chamber of the heart.

Another aspect of the invention provides for a method for deploying two or more implantable devices at a target site using a deployment system comprising a cannula and a deployment rod. Each device is releasably attached to a deployment assembly disposed at the distal end of the deployment rod, and each device may comprise an attachment element. The deployment assembly may comprise first and second shrouds.

Unless otherwise qualified, the term “pair”, such as “a pair of small implantable elements” is intended to mean two or more items. The term “about” with reference to a value (e.g. “about 10 mm”) is intended to mean that the value may vary in a range of ±10% around the value, or in a range of ±20%. Thus, a value of “about 10 mm” may vary between a range of 9-11 mm, or in a range between 8-12 mm.

A deployment system of the present invention generally includes an introducer cannula, a deployment rod, a deployment assembly, and a device. The direct deployment system may further comprise a needle disposed within a cannula (“needle-core”) or separate from the cannula. Unless otherwise specified, any reference to “cannula” shall refer to both needle-core cannulas and non-needle-core cannulas. The introducer cannula comprises an interior lumen that houses the deployment assembly in addition to the deployment rod, once the deployment rod is inserted at the proximal end of the cannula. The deployment rod is used to advance the deployment assembly (with the attached device) through the cannula to the target site and to withdraw the deployment assembly once the implant is securely embedded. In addition to advancing and withdrawing the deployment assembly, the deployment rod may also be rotated, for example, clockwise or counterclockwise during positioning of the device.

The deployment rod will typically have an elongated and narrow profile so as to fit within the catheter and thereby permit percutaneous delivery. The deployment rod may have a uniform or non-uniform diameter over its length. Exemplary but non-limiting embodiments of a deployment rod include a pushrod, tube, hypotube, and wire.

The device may include a sensor that can monitor or detect any suitable physical, chemical, or biological characteristic of a body, e.g., a pressure, velocity, temperature, pH, a biological molecule, and/or an antigen. The sensor may be any sensor as is known in the art that would be suitable for measuring a characteristic of the body. In one embodiment, the characteristic has clinical value to a practitioner. When multiple sensors are positioned in the body, each sensor can measure the same parameter or different parameters, such as (but not limited to) temperature, pressure, and flow. The sensor may have a fill port to fill the sensor with a fluid, or may comprise a protruding tab which may extend through an aperture in a locking plate. Exemplary embodiments of vibrating or resonating sensors which may be used with the present invention are disclosed in U.S. Pat. Nos. 5,989,190; 6,083,165; 6,331,163; 7,415,883; and 8,162,839, all to Kaplan; U.S. Pat. No. 7,134,341 to Girmonsky et al.; and U.S. Pat. No. 10,105,067 to Richter et al.

The sensor may be attached to a locking plate via a fixation element (e.g., a clip, screw, ring, welding, and/or adhesive) and the locking plate may be affixed to a base. Alternatively, the sensor may be prepared as a single unit with the base and locking plate. The locking plate may include features to facilitate locking engagement with the deployment assembly. For example, the locking plate may include one or more (e.g., one, two, three, four, or more) indented portions around the circumference of the locking plate in any suitable size and shape to facilitate locking engagement with the deployment assembly. One or more of the indented portions may comprise a different shape than the other indented portions.

Similarly, the base may include features to facilitate locking engagement with the deployment assembly. For example, the base may include one or more (e.g., one, two, three, four, or more) cutout portions around the circumference of the base in any suitable size and shape to facilitate locking engagement with the deployment assembly. The indented features of the locking plate and the cutout portions of the base may have complementary shapes and sizes. For example, the indented portions and/or the cutout portions may be generally rectangular or arcuate. In alternative embodiments, instead of an indentation/cutout combination for locking engagement, a protrusion/complementary runner combination may be used. For example, a protrusion in the form of a pin, tab, or similar structure may be used in conjunction with a runner or other structure (similar to a clasp on an umbrella) to facilitate locking engagement. Other structures which provide equivalent locking engagement may also be used.

The device may further include an attachment element coupled to the base for anchoring the sensor into a vessel lumen or bodily tissue. The attachment element may include a structure such as, e.g., a barb, hook, latch, ring with legs, screw, spear, or tack, or other structure which secures the device in the target location. The distal-most end of the attachment element may include a sharpened tip to facilitate penetration into the target vessel or tissue. The attachment element, base, and locking plate may be made of stainless steel, titanium, MP35N or other suitable nickel-chromium alloy, L-605 or other suitable cobalt-chromium alloy, a biocompatible polymer, a shape-memory alloy (e.g., Nitinol), or other suitable material as is known in the art. Exemplary embodiments of attachment elements are illustrated in US 2014/0012101, published on Jan. 9, 2014, the contents of which are incorporated herein by reference in their entirety.

The deployment assembly at the distal end of the deployment rod provides an operator-controlled, releasable attachment for the device and generally includes a shroud. The shroud may include one or more (e.g., one, two, three, four, or more) optional circumferential securement tabs for engaging with the cutout portions of the base and locking plate thereby securing the device. The circumferential securement tabs couple the device to the deployment assembly and the deployment rod and thus transmit the forces from the deployment rod to the device.

In particular embodiments, the deployment assembly may further comprise a release member. The release member has a structure which maintains the implantable device releasably attached to the shroud during implantation. There may be any number of release members, for example, one, two, three, four, or more. After the device is securely positioned at the target site, the release member may be disengaged so as to release the shroud from the device. In one embodiment, the release member may have an elongate structure, such as a release wire as further discussed. In an exemplary embodiment, the release member is a release wire having a bifurcated distal end, and disengagement of the release wire uncouples the shroud from the device so that the device separates from the shroud. In other embodiments, the release member may be a pin, lock, latch, spring, or other element(s) which releasably couples the device to the shroud. The release member may have a unitary structure, or the release member may comprise distinct elements which are assembled to provide a structure which maintains the device and shroud together during deployment and releases the device from the shroud upon an operator's action, such as application of a negative force (a pulling action) or a positive force (a pushing action).

The shroud may further include one or more (e.g., one, two, three, four, or more) axial securement tabs for securing the device from axial motion (i.e., axially along the catheter lumen) that could cause premature detachment. The axial securement tabs may be flexible and/or moveable between two configurations such that the tabs may be in a first, locked (i.e., engaged) configuration or in a second, unlocked configuration allowing for axial movement of the tabs and thereby detachment of the device upon secure positioning. In other exemplary embodiments, the deployment assembly may detach the device after positioning via a rotational movement, such as by unscrewing or turning after the securement tabs are released.

The shroud may have a diameter in the range of about 0.1 mm to about 20 mm and a cross-sectional thickness in the range of about 0.01 mm to about 5 mm. In an exemplary embodiment, the diameter of the shroud is about 4 mm and the thickness is about 0.4 mm. The axial securement tabs may each have a length of about 0.05 mm to about 50 mm and a width of about 0.05 mm to about 20 mm. In one exemplary embodiment, the length of each axial securement tab is about 6.6 mm and the width is about 1.4 mm. The optional circumferential securement tabs may each have a length of about 0.05 mm to about 50 mm and a width of about 0.05 mm to about 20 mm. In one embodiment, the length of each circumferential securement tab is about 2.2 mm and the width is about 1.5 mm. The tabs may have similar or different structures and configurations.

In an embodiment of the invention, a release member may be coupled to the shroud such that, until withdrawn, the axial securement tabs remain in the locked configuration, thereby preventing detachment of the device from the deployment assembly. Once the release member is disengaged by an operator, for example, by pulling the member, the axial securement tabs may assume an unlocked configuration upon withdrawal of the deployment rod such that the deployment assembly separates from the device. In one embodiment, the deployment assembly separates from the device upon reaching a predetermined limit such as a force limit or depth limit.

The shroud and release member may be made of a metal, e.g., stainless steel, titanium (pure or alloys), cobalt-based alloys (e.g., MP35N or L-605), tantalum, zirconium, platinum (pure or alloys), gold (pure or alloys), a biocompatible polymer, a shape-memory alloy (e.g., Nitinol or superelastic Nitinol), or any suitable polymer, e.g., polyurethane (PU), polyethylene terephthalate (PET), polyethylene (PE), polyamide, polyimide, polyether ether ketone (PEEK), polyglycolic acid, polyetherimide (ULTEM), or polylactide, or another suitable material, or a combination of materials as is known in the art. Certain structural features of the shroud or release member may be formed of one type of material while other components may be formed of another type of material having different properties, for example, greater flexibility, as may be desirable.

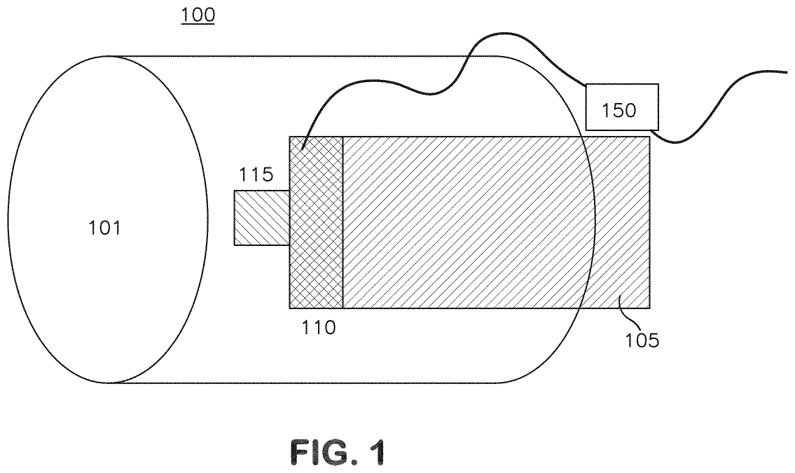

FIG. 1 illustrates a block diagram of a deployment system 100, whereby a deployment rod 105 in the form of a pushrod is located in the interior lumen of introducer cannula 101. Deployment assembly 110 is located at the distal-most end of the deployment rod 105, with device 115 attached to the distal-most end of the deployment assembly 110. The deployment assembly may optionally further comprise a force meter 150 to provide feedback to the operator regarding measurements of the pushing force used to embed the device 115 and/or the pulling force applied to an embedded device.

The introducer cannula is adapted to house the deployment rod, the deployment assembly, and the device. Optionally, a needle-core cannula may be adapted to house a needle wherein the needle can be retracted through the cannula after initial tissue piercing and/or during transport of the device to the implantation site. The cannula may comprise an outer diameter in the range between 1 to 50 G, an inner diameter in the range of 0.1 to 20 mm, a length of 1 to 200 cm, and comprises a suitable flexible, semi-flexible, or rigid biocompatible material for use within the body. Suitable materials include, for example, silicones, polyvinyl chloride (PVC) or other medical grade biocompatible polymers. In one particular embodiment, the introducer cannula has an outer diameter of 17 G, an inner diameter of 1.06 mm, a length of 20 cm and is made of a semi-flexible, biocompatible material. In another exemplary embodiment, the introducer cannula has an outer diameter of about 5 mm, an inner diameter of about 4 mm, and a length of about 105 cm.

The deployment rod is contained within the interior lumen of the introducer cannula and is attached to the device via the deployment assembly. The dimensions of the deployment rod will depend upon the particular implementation, and in an exemplary embodiment, the deployment rod may have an outer diameter in the range of about 0.1 mm to about 40 mm, and a length in the range of about 0.1 to about 500 cm. The deployment rod may be hollow and in one embodiment, have an inner diameter of about 0.1 mm to about 2 mm defining a lumen. In an embodiment of the invention, a release member (such as a release wire described in more detail below) may pass through the lumen.

The deployment rod may be adapted to move or advance lengthwise inside the lumen of the cannula from the proximal end of the cannula to the target implantation site to deploy the implantation device. The deployment rod may also be adapted to be rotated clockwise or counterclockwise to securely position the device into the target site. In the embodiment where the deployment rod is adapted to rotate, the deployment rod may be a torque coil. A torque coil comprise a helical coil spring having tightly packed coils or, alternatively, may have a predetermined pitch space between the coils. For example, a rigid material may be preferred in the case of needle-based delivery. The deployment rod may be formed of a suitable biocompatible material which may be rigid or semi-flexible, such as but not limited to silicone, polyvinyl chloride (PVC), polyether ether ketone (PEEK), polyetherimide (ULTEM), titanium (pure or alloys), cobalt-based alloys (e.g., MP35N or L-605), Nitinol, tantalum, zirconium, platinum (pure or alloys), gold (pure or alloys), or stainless steel. The materials of the cannula and the deployment rod may be the same or different.

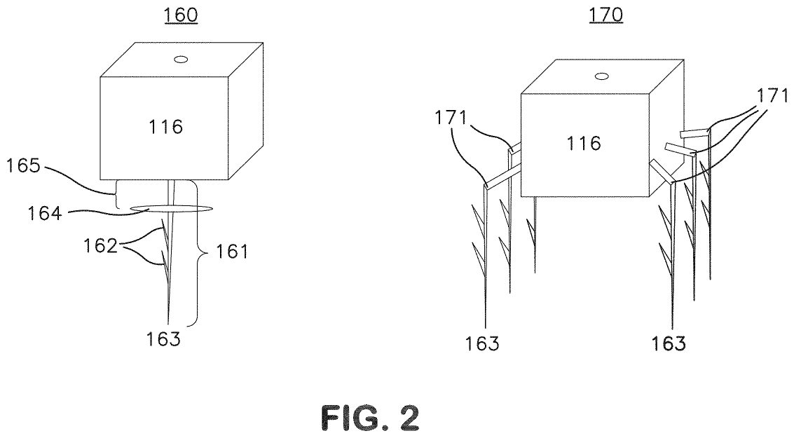

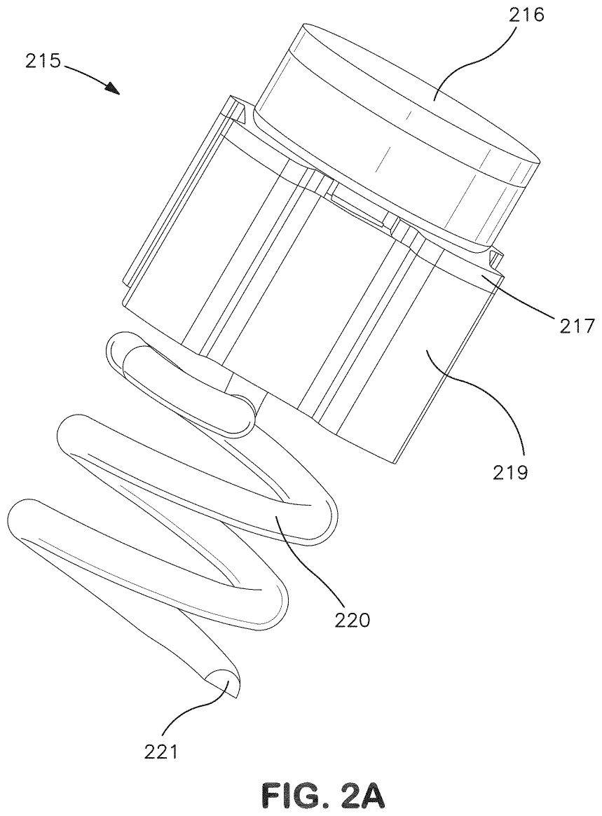

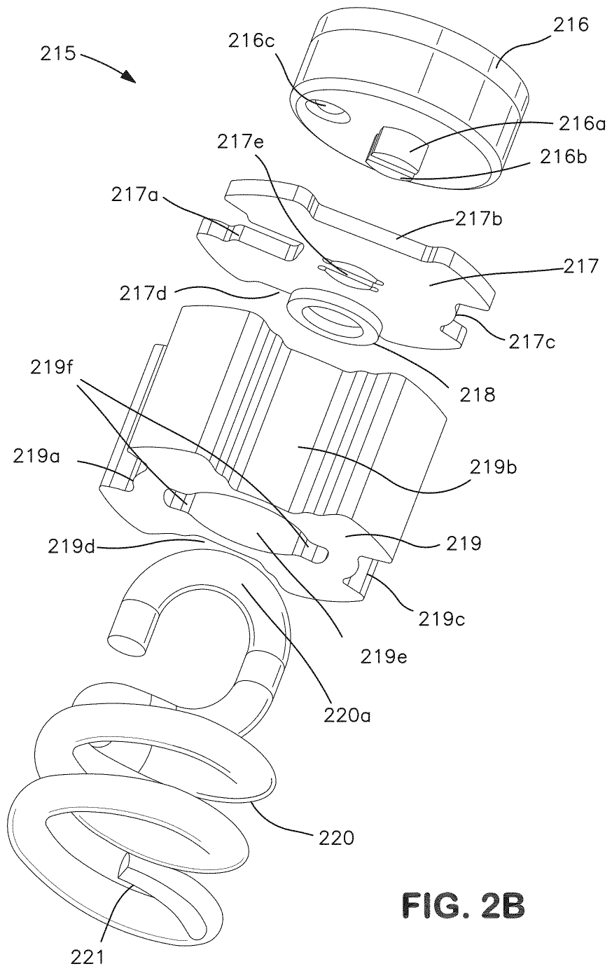

FIG. 2 illustrates embodiments of implantable devices 160 and 170 having attachment elements in the form of spears 163. In one embodiment, a device 160 includes a sensor 116 and a spear 163 with optional barbs 162 to ensure secure positioning in a target site such as a vessel wall or tissue. The spear 163 has a tip which penetrates the target site and may optionally have one or more barbs 162. The spear 163 may optionally have a stop 164 which prevents the entire length 161 of the spear 163 from being implanted into the target tissue, leaving a gap 165 between the stop 164 and the sensor 116. In an alternative embodiment, the device 170 may comprise a sensor 116 having a plurality of spears 163 such as, e.g., six spears as illustrated. The device 160, 170 may have any number of spears 163 (or other attachment elements), such as one, two, three, four, five, six, or more, and they may be located at any suitable position. In further embodiments, the spears 163 may optionally have hinges 171.

FIG. 2A illustrates a device 215 having a coiled attachment element 220. In FIG. 2A, the device 215 includes a sensor 216 that is coupled to a locking plate 217 via a fixation element (not shown). The locking plate 217 is attached to a base 219 that is coupled to an attachment element 220 having a helical coil. In alternative embodiments of the invention, any of the locking plate 217, base 219, and attachment element 220 may be joined as a single element. For example, the locking plate 217 and base plate 219 may be formed as a single structure. The helical coil has a sharpened distal-most tip 221 for improved penetration of a vessel wall or tissue. For example, the attachment element may be ground down or cut at the distal-most tip in such a way to create a sharp end or a smaller end. In another embodiment, a coil having a decreasing diameter may be used such that the coil has a decreasing diameter towards the distal-most end. In alternative embodiments, the attachment element may be a barb, hook, latch, ring with legs, screw, spear, tack, or other structure which fixes the device in the target location.

FIG. 2B illustrates an exploded view of a device 215 having a coiled attachment element 220. It is to be understood that in other embodiments of the invention, even if not expressly stated, the attachment element 220 may be a hook, spear, or another structure as discussed previously. A coiled attachment element 220 may have any number of turns so that the device 215 is securely embedded in the target location. As described above, the device 215 includes a sensor 216 coupled to a locking plate 217 via a fixation element 218. It is to be understood even if not expressly stated that the locking plate 217, base 219, and attachment element 220 may be provided as a single unit or as individual units which are assembled to form the desired structure.

The sensor 216 includes two protruding tabs 216a, 216b that may be positioned in an aperture 217e of the locking plate 217. The sensor 216 may include a fill port 216c for filling the internal volume of the sensor with a fluid, such as water. The fill port 216c may be sealed with a sealing ball to prevent fluid from flowing out of the inside compartment of the sensor 216. The fixation element 218, e.g., a ring, may be tightly secured over the tabs 216a, 216b to thereby couple the sensor on the locking plate 217. Alternative fixation elements which fix the sensor 216 to the locking plate 217/base 219, such as but not limited to adhesives and welding are possible and within the scope of the present invention. Such alternative fixation elements may replace elements such as 216a, 216b, and 218, and/or others as appropriate. The locking plate 217 further includes indented portions 217a-217d disposed about the circumference of the plate 217 and may be any suitable size and shape. In particular, all indented portions 217a-217d are generally rectangular in shape with rounded edges, but indented portion 217a is a different size (i.e., larger) than indented portions 217b-217d. Indented portion 217a may be sized differently (e.g., larger) to provide access to the fill port 216c of the sensor 216. The indented portion 217a may also provide space for the sealing ball of the fill port 216c to protrude outwardly.

The locking plate 217 is coupled to a base 219 via methods known in the art, such as welding or adhesives. For example, the locking plate 217 may be coupled to the base 219 by laser welding. Similar to the locking plate 217, the base 219 includes cutout portions 219a-219d disposed about the circumference of the base 219 and may be any suitable size and shape. In particular, all cutout portions 219a-219d are generally rectangular in shape with rounded edges, but cutout portions 219b and 219d are a different size (i.e., larger) than cutout portions 219a and 219c.

An attachment element 220 is coupled to the base 219 within an aperture 219e. In one embodiment, the attachment element 220 is coupled to the base 219 via a proximal loop 220a, although other types of connections are within the scope of the present invention. The proximal loop 220a may be rotatably fixed within a slotted cavity 219f within the aperture 219e. The attachment element 220 may alternatively be coupled to the base via, e.g., laser welding. In one embodiment, the attachment element 220 is a helical coil having two or more loops of wire and a distal-most tip 221 that is partially ground down or pointed to improve penetrability of the tip 221 into a vessel wall or tissue.

FIG. 2B illustrates one embodiment of a coupling between the base 219 and the attachment element 220. In other embodiments, the attachment coil 220 may not be coupled directly to the base 219 itself (where the proximal loop 220a is assembled in the aperture 219f), but instead may be coupled through an intermediate structural element, such as, but not limited to, a shaft which can be a rigid or flexible hollow or solid tube. The attachment element 220 may be coupled distally to the base 219 via welding of a proximal geometric feature in the coil, but the part which the coil will be coupled to may vary. In certain embodiments, the attachment element 220 and the base 219 may be manufactured as a single unit, in which case no coupling would be needed. In further embodiments, the coil may not have a proximal loop 220a but another shape which is not a loop. In alternative embodiments, the attachment element may be a barb, coil, hook, latch, ring with legs, screw, spear, tack, or other structure.

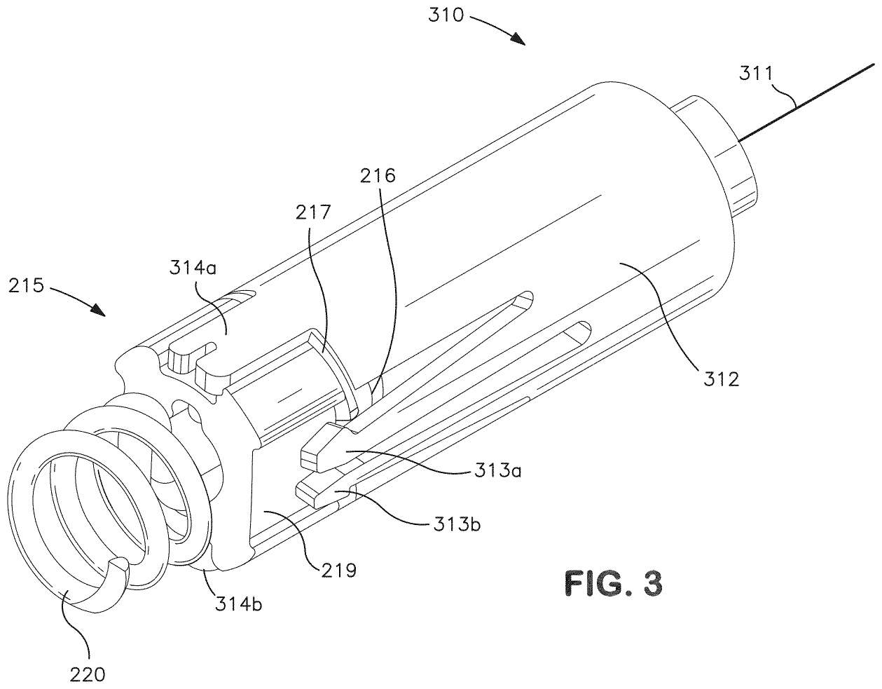

FIG. 3 illustrates a deployment assembly 310 releasably attached to a device 215. The device 215 is similar to the device described above in FIGS. 2A and 2B and similarly includes a sensor 216, a locking plate 217, a base 219, and a coiled attachment element 220. As shown in FIG. 3, the deployment assembly 310 includes a release member in the form of a release wire 311 and a shroud 312 that together allow for operator-controlled release of the device 215. The shroud 312 includes axial securement tabs 313a, 313b and optionally circumferential securement tabs 314a, 314b as shown in FIG. 3. In an alternative embodiment, a single set of tabs can provide both axial and circumferential securement. In one embodiment, the axial securement tabs are positioned opposite one another around the circumference of the shroud 312 and are generally long and thin cantilevered tabs extending distally from the shroud 312 that are capable of deformation when a predetermined force is applied.

When the device 215 is attached to the deployment assembly 310, the axial securement tabs 313a, 313b engage with the cutout portion of the locking plate 217 to thereby prevent axial motion of the device 215. In one embodiment, the circumferential securement tabs 314a, 314b are positioned opposite one another around the circumference of the shroud 312 and have similarly cantilevered tabs extending distally from the shroud 312. The axial length of the circumferential securement tabs 314a, 314b may be the same or different than the length of the axial securement tabs 313a, 313b.

The release wire 311 is coupled to the shroud 312 as a “safety” mechanism to prevent premature deployment of the device 215. To prevent premature release of the device 215, the release wire 311 is coupled to a structure that is inserted in between axial securement tabs 313a and 313b to prevent premature deformation of the tabs towards one another. Consequently, deformation of the tabs 313a and 313b does not occur and the locking plate 217 of the device 215 is secured from axial motion that would separate the device 215 from the deployment assembly 310. In another exemplary embodiment (not illustrated), the distal end of the release wire 311 may be looped around the axial securement tabs 313a, 313b, such that a pulling action on the release wire 311 will create a tension on the axial securement tabs 313a, 313b and cause them to deform and move together. This deformation allows the axial securement tabs 313a, 313b to pass over the locking plate 217 as the release wire 311 is pulled, thereby uncoupling the implanted device 215 from the shroud 312. In a further embodiment, a negative force such as a pulling action on the deployment rod (not illustrated in FIG. 3), may be sufficient to cause the axial securement tabs 313a, 313b to deform and disengage the device 215 from the shroud 312, without the presence of an additional structural element such as a release member.

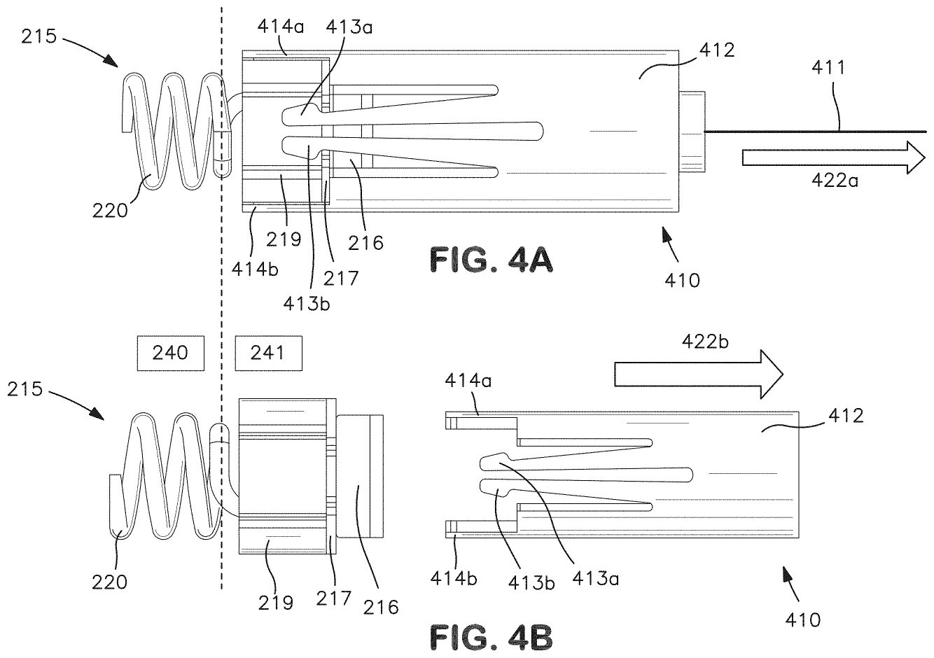

FIG. 4A illustrates a first step in releasing the device 215 from a deployment assembly 410. The dashed line in FIG. 4 represents the tissue wall 240 relative to the interior open chamber 241 of the heart. The device 215 is similar to the devices as shown in FIGS. 2 and 3 and includes a sensor 216, a locking plate 217, a base 219, and an attachment element 220. The deployment assembly 410 is similar to the deployment assembly shown in FIG. 3 and includes a release wire 411 and a shroud 412 having axial securement tabs 413a, 413b and circumferential securement tabs 414a, 414b.

As shown in FIG. 4A, the device 215 is securely embedded within the tissue wall of a chamber of the heart and the sensor 216, locking plate 217, and base 219 are protruding in the heart chamber. The shroud 412 is coupled to the device 215 via the axial securement tabs 413a, 413b and optionally by the circumferential securement tabs 414a, 414b. In one embodiment, as the shroud 412 is retracted, the axial securement tabs 413a, 413b deform towards one another as they pass over the locking plate 217 of the device 215. Removing the release wire allows the axial securement tabs 413a, 413b to be deformed in order to allow retraction of the shroud 412 to separate the shroud 412 from the device 215.

In the first step to release the device 215, an operator applies a pulling force (i.e., a negative force) 422a to the release wire 411 to thereby retract or otherwise release a safety mechanism (not shown in this figure) from between the axial securement tabs 413a, 413b. If the pulling force to the release wire 411 is not applied, the axial securement tabs 413a, 413b cannot move towards one another to allow the locking plate 217 to clear the tabs 413a, 413b. There may be one or more safety mechanisms to prevent premature detachment of the device 215 from the shroud 412 with the predetermined force limit.

FIG. 4B illustrates a second step in releasing the device 215 from the deployment assembly 410 wherein the deployment assembly 410 is separated from the device 215. In particular, after the release wire 411 is retracted, an operator will separate the device 215 from the deployment assembly 410 by retracting the deployment rod connected to the shroud 412.

As shown in FIG. 4B, after the release wire 411 is pulled out of the shroud 412, the deployment rod may be retracted in the proximal direction 422b to complete separation of the shroud 412 from the device 215. The shroud 412 generally remains affixed to the distal end of the deployment rod to facilitate removal of the shroud from the patient's body during the surgical procedure. In another embodiment, the retraction of the shroud 412 can be performed while applying a counterforce to the implant via another element (not shown). This counterforce reduces the net force on the implant which minimizes the chances of dislodgement from the tissue during positioning and delivery separation. The deployment rod and cannula are then retracted fully out of the patient's body.

In another embodiment, a pulling force (i.e. a negative force) may be sufficient to release the device 215 from the shroud 412 without the presence of a release member, release wire, or other structure inserted in between axial securement tabs 413a and 413b. In this embodiment, application of a sufficient pulling force on the deployment rod (not illustrated in FIGS. 4A and 4B) will cause the shroud 412 to move in a proximal direction. This pulling force will cause the securement tabs 413a, 413b to deflect and move together as they go past the locking plate 217 while the implantable device is positioned in the target location. The retraction of the shroud 412 can be performed while applying a counterforce to the implant via another element (not shown). As discussed above, this counterforce reduces the net force on the implant which minimizes the chances of dislodgement from the tissue during positioning, delivery, or separation.

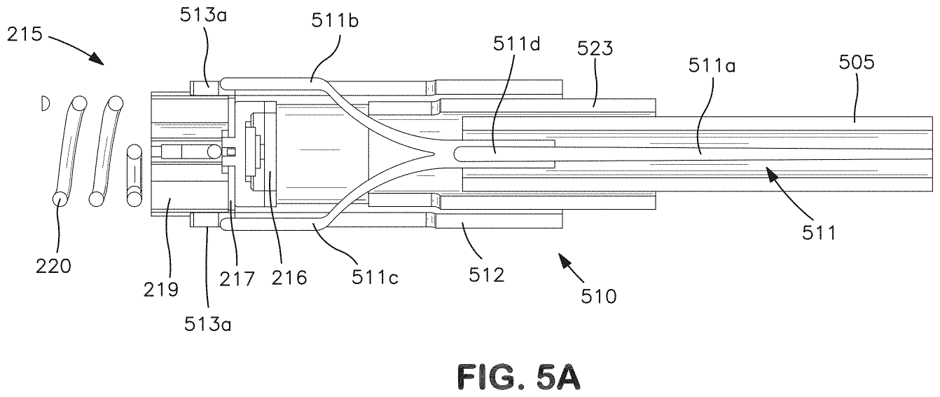

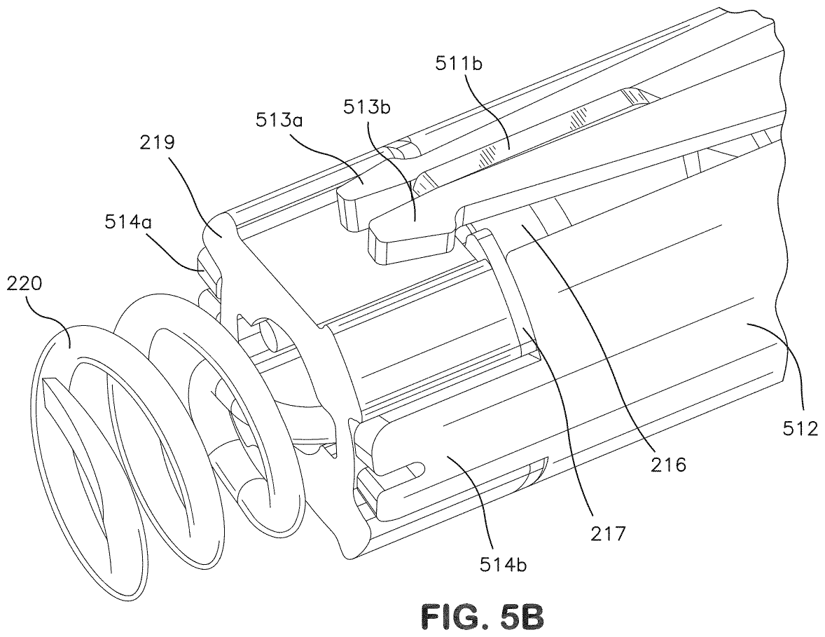

FIG. 5A illustrates another embodiment as a cross-sectional view and FIG. 5B illustrates a top view of a deployment assembly 510 attached to a device 215 and releasably coupled to a release wire assembly 511. The device 215 is similar to the devices as shown in FIGS. 2, 3, and 4A-4B and includes a sensor 216, a locking plate 217, a base 219, and an attachment element 220. The deployment assembly 510 includes a deployment rod 505, release wire assembly 511 and a shroud 512 having axial securement tabs 513a (513b not shown in FIG. 5A).

A tubular adapter part 523 is used to match the assembly diameter of the shroud 512 to the diameter of the deployment rod 505. In the embodiment illustrated in FIG. 5A, the shroud 512 and the deployment rod 505 have different diameters and therefore the adapter part 523 is used in the assembly to compensate for the gap in diameters between the shroud 512 and deployment rod 505. The adapter part 523 may be welded to the shroud 512. In other embodiments in which the shroud 512 and deployment rod 505 fit together without a gap, an adapter part 523 would not be necessary and the shroud 512 may be welded directly to the deployment rod 505. The shroud 512 may be constructed from either a single part or from two (or more) parts fixed together.

As shown in FIG. 5A, the release wire assembly 511 includes a release wire 511a and two bifurcated wires 511b, 511c. The bifurcated wires 511b, 511c are coupled to the release wire 511a via a coupling 511d, which may be, for example, a joint. That is, the bifurcated wires may be welded to the release wire. In an alternative embodiment, the bifurcated wires and the release wire are part of a single structural element and prepared using e.g. laser cutting. The bifurcated wires 511b, 511c extend outwardly from the coupling 511d towards the outer circumference of the shroud 512 where each bifurcated wire 511b, 511c is positioned between axial securement tabs 513a, 513b (513b out of view in FIG. 5A). The bifurcated wires 511b, 511c therefore serve as a retractable safety mechanism to prevent premature separation of the securement tabs 513a, 513b from disengaging from the plate 217. Because the bifurcated wires 511b, 511c are positioned between the axial securement tabs 513a, 513b as shown in FIG. 5B, the axial securement tabs 513a, 513b cannot move towards one another to allow the locking plate 217 to clear the tabs 513a, 513b and thus separate the device 216 from the deployment assembly 510.

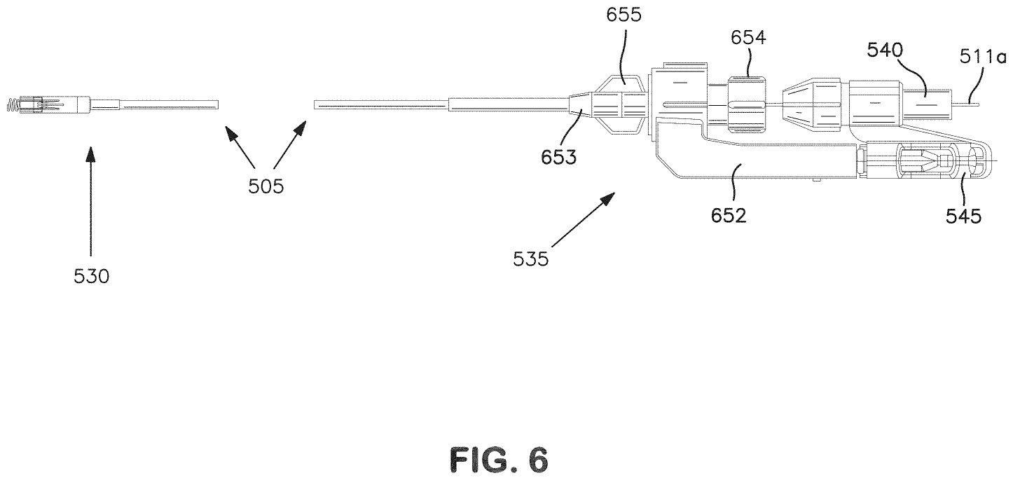

FIG. 6 illustrates separate distal 530 and proximal 535 sub-assemblies in an embodiment of a deployment system. The distal sub-assembly 530 and proximal sub-assembly 535 are connected via a deployment rod 505. The distal sub-assembly 530 comprises an implant and release mechanism, and the proximal sub-assembly 535 comprises a handle 652 with a safety clip 550 coupled to a release wire 511a. The handle 652 is connected to a distal cup 653 and a cuff 654. The distal cup 653 is fixedly attached to the deployment rod 505 and has a plurality of fins 655 which can be used to facilitate rotation of the distal cup and thereby the deployment rod 505 and the distal sub-assembly 530. Such rotation may be desirable in order to facilitate implantation of the device at the target location as discussed earlier. In other embodiments, the position of the cuff 654 can be adjusted laterally or rotationally with respect to the handle to allow for lateral movement of the deployment rod to facilitate positioning of the device during deployment. In other embodiments, both the distal cup 653 and the cuff 654 may be configured for lateral and/or rotational movement to facilitate deployment of the implantable device 115, 215. The distal cup 653 and the cuff 654 may have their own locking elements (not illustrated) to prevent undesired movement during implantation. In certain embodiments, it may also be desirable to turn or rotate the handle during deployment of the device.

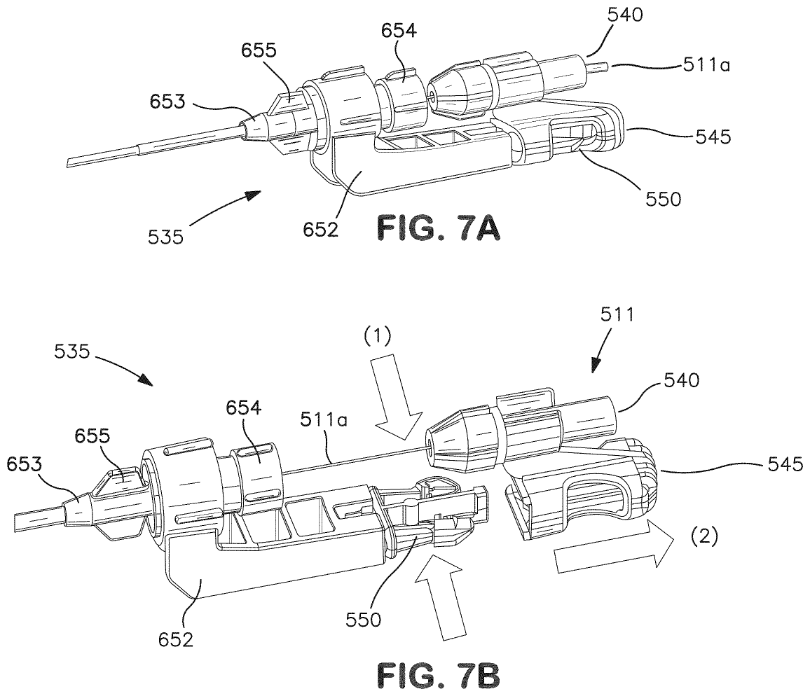

FIGS. 7A and 7B illustrate a proximal sub-assembly 535 comprising a handle 652, distal cup 653, cuff 654, wire connector 540, safety clip 550, and holder 545. The release wire 511a, which may extend from the distal end to the proximal end of the deployment system, is coupled to the handle 652 via a wire connector 540 (such as a torquer or other element having the same function) which grips the thin release wire on its diameter and keeps it in tension through the shaft of the delivery system. The wire connector 540 is connected to the handle 652 via a holder 545.

The proximal sub-assembly 535 also comprises a lock assembly to prevent unintended or premature disengagement of the release wire 511a. In FIGS. 7A and 7B, the lock assembly comprises a clip 550 and holder 545. The lock assembly may comprise any suitable structure or elements such as a pin or clip (as illustrated). In FIGS. 7A and 7B, the safety clip 550 has two flexible lateral tabs, one on each side of the handle 652. The safety clip 550 releasably secures the holder 545 in place. The holder 545 is also coupled to the release wire 511a through the wire connector 540 thereby providing a wire holder assembly. The deployment assembly 510 prevents the release wire assembly 511 from being pulled prematurely.

After the device 215 is positioned at the target location, the device 215 is disengaged from the deployment assembly 510. In operation, the safety clip 550 is first disengaged by the operator. To disengage the safety clip 550, the opposing lateral tabs of the safety clip 550 are pressed (for example, with the index finger and thumb). This action causes the holder 545 to disengage from the safety clip 550. At this stage, the entire release wire assembly comprising the wire connector 540, holder 545, and the release wire 511a itself can be gripped by hand and retracted proximally. As the release wire 511a is retracted, the bifurcated wires 511b, 511c also retract proximally through the deployment rod 505 in a deformed shape. This retraction of the release wire 511a disengages the bifurcated wires 511b, 511c from the axial securement tabs 513a, 513b in the shroud 512 at the distal end of the deployment system. In certain embodiments, a “clicking” movement may occur when the release wire 511b, 511c disengages from the tabs 513a, 513b of the shroud 512 to provide a sensory indication that the device 215 has disengaged from the deployment assembly 510. The shroud 512 is now free to move away (proximally), or be unscrewed or otherwise detached from the device 215, and the entire deployment system can be completely retracted from the device 215 and out of the patient's body, leaving the implanted device 215 in the target location.

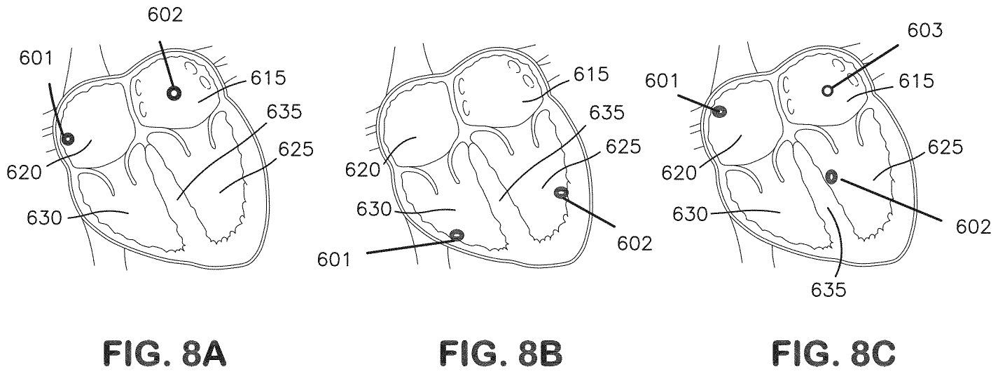

FIG. 8A shows in one embodiment a first sensor 601 deployed in the right atrium 620 of the heart and a second sensor 602 deployed in the left atrium 615 of the heart in an embodiment of the invention. The sensors 601, 602 can be deployed in the heart in any particular order or placement in accordance with a clinician's expertise. As illustrated in this embodiment, the first sensor 601 may be deployed in the right atrium 620 and subsequently the second sensor 602 may be deployed in the left atrium 615, or the order may be reversed so that the first sensor 601 is deployed in the left atrium 615 and the second sensor 602 is deployed in the right atrium 620. The sensors may be deployed at any location in the target location, such as on the chamber wall or septum. As shown in FIGS. 4A and 4B, after deployment the attachment element will be positioned in the tissue wall 240 and the sensor will be located in the heart chamber 241 (or other location) for monitoring the patient's physiological conditions.

FIG. 8B shows in another embodiment a first sensor 601 positioned in the right ventricle 630 of the heart and a second sensor 602 positioned in the left ventricle 625 of the heart, in an alternative embodiment of the invention. FIGS. 8A and 8B show a sensor 601, 602 positioned in each atrium chamber 615, 620 or in each ventricle chamber 625, 630, although the sensors may be positioned in any desired location in the body.

FIG. 8C shows in yet another embodiment of the invention three sensors 601, 602, 603 are positioned in the heart. In the illustrated embodiment, a first sensor 601 is positioned in the right atrium 620 of the heart, a second sensor 602 is positioned in the left ventricle 625 of the heart on the ventricular septum 635, and a third sensor 603 is positioned in the left atrium 615 of the heart. The invention is not restricted as to the particular placement of one or more sensors in the body, and it will be evident that the sensors can be positioned at any target location to provide data concerning the physiological conditions at the implant location.

Another aspect of the invention is directed to methods of monitoring physiological conditions provided by two or more sensors positioned at a target location in a patient's body. This aspect of the invention can be used in conjunction with the sensors described above in order to provide physiological data to a medical practitioner.

After a sensor is positioned at a target location, it will be necessary to monitor the readings from the sensor. If there is only a single sensor, for example, a sensor which changes its resonance frequency as a function of ambient pressure, readings from the sensor are generally straightforward to obtain and interpret. However, when there are two (or more) sensors positioned near each other, for example, one sensor in each atrium or ventricle of the heart, it is necessary to identify which sensor is giving a particular reading.

When a clinician intends to place different sensors in target locations with different ambient pressures, such as for measuring blood pressure in the heart, the clinician will need to ensure there is no coupling between the sensors, or that any coupling is small enough to allow each sensor to respond independently of other sensor to pressure changes at the respective implantation site. It was not previously known how to measure physiological conditions provided by two or more sensors positioned at different locations in the body.

The invention also includes a method for measuring physiological data provided by multiple implanted sensors using frequency separation and spatial separation. The inventive methods are discussed below with particular reference to pressure sensors, but the methods described can be used for measuring other kinds of physiological conditions using other kinds of sensors and therefore are within the scope of the present invention. Each of the sensors may have an attachment element such as a barb, coil, hook, latch, ring with legs, screw, spear, and tack, or another structure suitable for maintaining the device at the desired location in the body.

Although the discussion below may specifically describe monitoring two sensors, the invention is equally applicable to monitoring three or more sensors in accordance with the principles described. Exemplary embodiments of vibrating or resonating sensors which may be used with the inventive methods described are disclosed in U.S. Pat. Nos. 5,989,190; 6,083,165; 6,331,163; 7,415,883; and 8,162,839, all to Kaplan; U.S. Pat. No. 7,134,341 to Girmonsky et al.; and U.S. Pat. No. 10,105,067 to Richter et al.

The frequency response of a particular sensor can be limited by fₘᵢₙ and fₘₐₓ i.e. for the r^{th} sensor, as shown in Equation 1:

fₘᵢₙⁱ≤fᵣⁱ≤fₘₐₓⁱ (eq. 1)

In order to measure frequency separation, frequency ranges of the different sensors should not overlap. In this way, all sensors located within the measuring probe beam can be measured simultaneously. Under certain clinical conditions, a difference in the measured property (e.g. blood pressure) in the two sensor locations will be present, with one sensor consistently measuring a higher value than the other sensor. For example, ventricles have thicker walls and generate higher blood pressures than atria. In such cases, the frequency separation can be achieved by ensuring the resonance frequencies that correlate to the measured properties can be differentiated by the measuring system. The use of clinical knowledge as to which sensor location would experience the higher value can be used to attribute a specific value to each sensor and thereby measure the frequency separation.

When the sensor has a nonlinear dependence and thus responds in higher harmonics, an additional condition as shown in Equation 2 can be used in case these harmonics are strong and interfere:

N*fₘᵢₙⁱ≤f_{rN}ⁱ≤N*fₘₐₓⁱ< >M*fₘᵢₙ^{j}≤f_{rM}^{j}≤M*fₘₐₓ^{j} (eq. 2)

where i and j are the sensor index and N and M are the harmonics number.

In one embodiment, physiological data such as pressure may be measured and transmitted using the method described in U.S. patent application Ser. No. 16,389,202, filed on Apr. 19, 2019 and entitled “Methods for the Use of Inherent Frequency Shifting Mechanisms for Sensors Response Reading With Continuous Wave Excitation”, incorporated herein by reference in its entirety.

In accordance with the spatial separation measurement technique, the spatial distance between the pair of sensors is large enough (that is, larger than the spatial resolution of the measurement probe) to allow the clinician to measure each sensor separately. Using, for example a standard ultrasound imager, a clinician may be able to visualize the different sensors in color Doppler mode. The clinician would then measure readings from a particular sensor by first steering the ultrasound beam such that only the single sensor passes through the beam axis and subsequently measuring the signals using a continuous wave Doppler spectral mode.

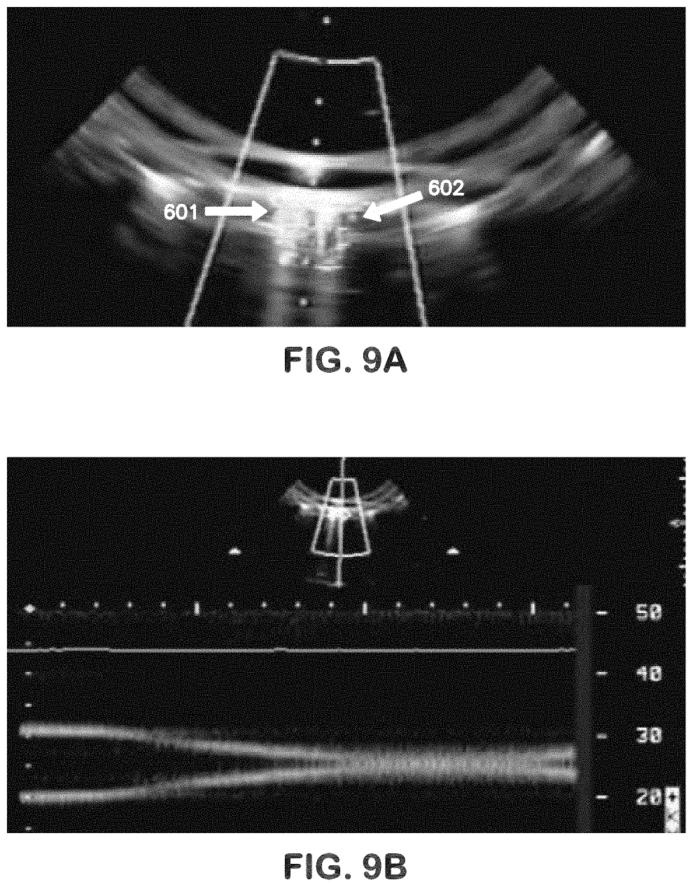

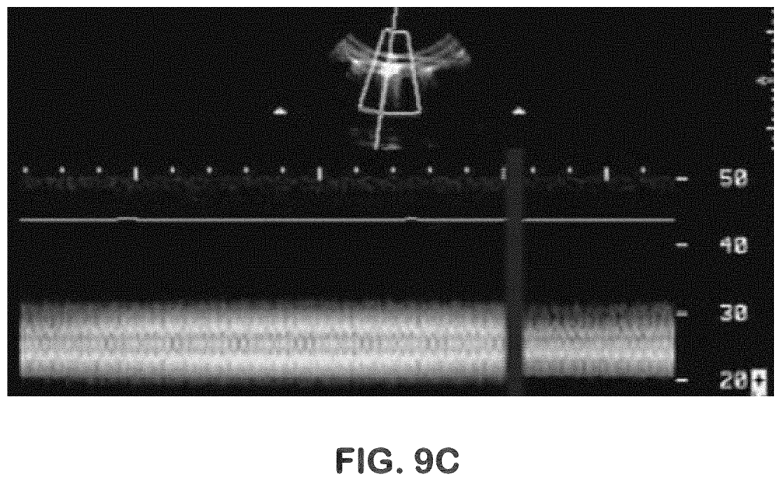

FIG. 9A is an ultrasound image of two sensors, a first sensor 601 and a second sensor 602, which are about 1 cm apart and seen as two colored “comet tails” in color Doppler mode obtained using a standard ultrasound imager. FIG. 9B shows the spectral response of the first sensor 601 from FIG. 9A via continuous wave Doppler mode. FIG. 9C shows the spectral response of the second sensor 602 from FIG. 9A via continuous wave Doppler mode. In FIG. 9C, the beam focus was shifted using a phased-array transducer. These FIGS. 9A, 9B, and 9C show that it is possible to determine which sensor is transmitting which signal and to measure each sensor separately.

Variations and modifications will occur to those of skill in the art after reviewing this disclosure. The disclosed features may be implemented, in any combination and subcombination (including multiple dependent combinations and subcombinations), with one or more other features described herein. The various features described or illustrated above, including any components thereof, may be combined or integrated in other systems. Moreover, certain features may be omitted or not implemented.

Examples of changes, substitutions, and alterations are ascertainable by one skilled in the art and could be made without departing from the scope of the invention disclosed herein. All references cited herein are incorporated by reference in their entirety and made part of this application.

What is claimed is:

1. A deployment system comprising:
a cannula;
a deployment rod having a proximal end and a distal end; and
an implantable device releasably attached to a deployment assembly disposed at the distal end of the deployment rod, said device comprising an attachment element,
wherein said deployment assembly comprises a shroud, said shroud releasably attached to the device.

2. The deployment system of claim 1, wherein the attachment element is selected from the group consisting of a barb, coil, hook, latch, ring with legs, screw, spear, and tack.

3. The deployment system of claim 1, wherein said deployment assembly further comprises a release member which disengages the device from the shroud.

4. The deployment system of claim 3, wherein the shroud is detached from the device upon retraction of the release member.

5. The deployment system of claim 1, wherein said release member comprises a release wire.

6. The deployment system of claim 5, wherein said release wire is bifurcated at a distal end.

7. The deployment system of claim 1, wherein said device further comprises a locking plate.

8. The deployment system of claim 7, wherein said locking plate comprises a plurality of indented portions.

9. The deployment system of claim 8, wherein one of said plurality of indented portions comprises a different shape.

10. The deployment system of claim 1, wherein said device further comprises a base having a distal side and a proximal side and a thickness in between, wherein the attachment element is affixed to the distal side of the base and the locking plate is affixed to the proximal side of the base.

11. The deployment system of claim 10, wherein said base comprises a plurality of cutout portions.

12. The deployment system of claim 11, wherein at least one of said plurality of cutout portions comprises a different shape.

13. The deployment system of claim 1, wherein said shroud comprises a plurality of axial securement tabs.

14. The deployment system of claim 13, wherein said plurality of axial securement tabs engage the locking plate to thereby prevent axial motion of the device with respect to the deployment assembly.

15. The deployment system of claim 1, wherein said shroud comprises a plurality of circumferential securement tabs.

16. The deployment system of claim 15, wherein said plurality of circumferential securement tabs engage the locking plate.

17. The deployment system of claim 1, wherein said device further comprises a sensor.

18. The deployment system of claim 17, wherein the sensor is affixed to a locking plate.

19. The deployment system of claim 17, wherein the sensor is configured to monitor a physiological parameter.

20. The deployment system of claim 19, wherein the physiological parameter is blood pressure.

21. The deployment system of claim 17, wherein the sensor is configured to monitor a chemical parameter.

22. The deployment system of claim 17, wherein said sensor comprises a fill port.

23. The deployment system of claim 17, wherein said sensor further comprises a protruding tab.

24. The deployment system of claim 23, wherein said protruding tab extends through an aperture in the locking plate.

25. A method for deploying an implantable device at a target site using a deployment system comprising a cannula and a deployment rod, said device releasably attached to a deployment assembly disposed at the distal end of the deployment rod, said device comprising an attachment element, said deployment assembly comprising a shroud and a release member, said method comprising:
advancing said deployment system to said target site such that the device is in contact with the target site;
securely positioning the attachment element in the target site using the deployment rod;
disengaging the release member and detaching a lock assembly to separate the shroud and device; and
withdrawing the deployment rod and cannula to separate the device from the shroud.

26. The method of claim 25, wherein the target site is a chamber of the heart.

27. The method of claim 25, wherein the attachment element is selected from the group consisting of a barb, coil, hook, latch, ring with legs, screw, spear, and tack.

28. The method of claim 25, wherein the step of securely positioning the attachment element in the target site comprises advancing the deployment rod.

29. The method of claim 25, wherein the step of securely positioning the attachment element in the target site comprises rotating the deployment rod.

30. The method of claim 25, wherein the deployment assembly further comprises a release member, and the step of disengaging the device from the shroud comprises disengaging the release member from the shroud and detaching a lock assembly.

31. The method of claim 30, wherein the step of disengaging the release member comprises administering a pulling force to the release member.

32. The method of claim 30, wherein the release member comprises a release wire.

33. The method of claim 30, wherein the release member comprises a release wire which is bifurcated at a distal end.

34. The method of claim 25, wherein the device comprises a sensor.

35. A method for deploying two or more implantable devices at a target site using a deployment system comprising a cannula and a deployment rod, wherein each device is releasably attached to a deployment assembly disposed at the distal end of the deployment rod, each device comprising an attachment element, said deployment assembly comprising first and second shrouds, said method comprising:
advancing said deployment system to a first target site such that a first device is in contact with the target site;
securely positioning the attachment element of the first device in the first target site;
disengaging the first device from the first shroud;
withdrawing the deployment rod and cannula to separate the first device from the first shroud;
advancing said deployment system to a second target site such that a second device is in contact with the second target site;
securely positioning the attachment element of the second device in the second target site;
disengaging the second device from the second shroud; and
withdrawing the deployment rod and cannula to separate the second device from the second shroud.

36. The method of claim 35, wherein the first and second devices are implanted at respective target sites during a single procedure.

37. The method of claim 35, wherein the first and second devices are implanted at respective target sites during separate procedures.

38. The method of claim 35, wherein the first and second target sites are left and right atria or left and right ventricles of the heart.

39. The method of claim 35, wherein the steps of securely positioning the attachment elements of the first and second devices comprise advancing the deployment rod.

40. The method of claim 35, wherein the steps of securely positioning the attachment elements of the first and second devices comprise rotating the deployment rod.

41. The method of claim 35, wherein deployment assembly further comprises first and second release members, and the steps of disengaging the first and second devices comprise disengaging first and second release members from first and second shrouds and detaching first and second lock assemblies.

42. The method of claim 41, wherein the steps of disengaging the first and second release members comprise administering a pulling force to the release members.

43. The method of claim 41, wherein the first and second release members are release wires.

44. The method of claim 35, wherein the first and second devices comprise first and second sensors.

45. The method of claim 35, further comprising the steps of:
advancing said deployment system to a third target site such that a third implantable device having an attachment element is in contact with the third target site;
securely positioning the attachment element of the third device in the third target site; and
separating the third device from a third shroud.

46. The method of claim 35, wherein each attachment element is independently selected from the group consisting of a barb, coil, hook, latch, ring with legs, screw, spear, and tack.

47. A method of monitoring physiological data, the method comprising:
deploying two or more small sensors at a target location in a patient's body; and
monitoring physiological data from each sensor using frequency separation or spatial separation.

48. The method of claim 47, wherein the sensors monitor blood pressure.

49. The method of claim 47, wherein the sensors monitor the same physiological property.

50. The method of claim 47, wherein the sensors monitor different physiological properties.

51. The method of claim 47, wherein the physiological data is monitored using continuous wave Doppler ultrasound imaging.

52. The method of claim 47, wherein the target location is the left and right atria or the left and right ventricles of the heart.

53. The method of claim 47, wherein each of the small sensors has an attachment element which embeds the sensor at the target location.

54. The method of claim 53, wherein each attachment element is independently selected from the group consisting of a barb, coil, hook, latch, ring with legs, screw, spear, and tack.

55. The method of claim 47, further comprising deploying a third small sensor device at a target location in the patient's body; and monitoring physiological data from the third sensor using frequency separation or spatial separation.
